(19) United States
(12) Patent Application Publication
(10) Pub. No.: US 2025/0312290 A1
(43) Pub. Date: Oct. 9, 2025

(54) COMPOSITIONS COMPRISING TERPENES

(71) Applicant: BUZZELET DEVELOPMENT AND TECHNOLOGIES LTD., Or Akiva (IL)

(72) Inventors: Noa RAZ, Gizo (IL); Aharon EYAL, Jerusalem (IL)

(21) Appl. No.: 18/865,694

(22) PCT Filed: May 15, 2023
(86) PCT No.: PCT/IB2023/054962

§ 371 Date: Nov. 14, 2024

Related U.S. Application Data
(60) Provisional application No. 63/342,355, filed on May 16, 2022.
(60) Provisional application No. 63/342,360, filed on May 16, 2022.
(60) Provisional application No. 63/342,362, filed on May 16, 2022.
(60) Provisional application No. 63/342,367, filed on May 16, 2022.
(60) Provisional application No. 63/342,373, filed on May 16, 2022.

(51) Int. Cl. A61K 31/015; A61K 9/20; A61K 31/00; A61K 31/045

(52) U.S. Cl. CPC A61K 31/015; A61K 9/20; A61K 31/045; A61K 31/658

(57) ABSTRACT

Provided are compositions comprising terpene mixtures consisting of 1000-4500 ppm myrcene, 1000-4000 ppm terpineol, 1000-5500 ppm caryophyllene and 1000-45000 ppm nerolidol; 1000-4500 ppm alpha-pinene, 1000-4000 ppm sabinene, 1000-4000 ppm limonene, 500-4000 ppm borneol and 1000-4500 ppm caryophyllene; 1000-4500 ppm alpha-pinene, 1000-4000 ppm linalool, 1000-4000 ppm terpineol, 1000-4000 ppm borneol, 1000-4500 ppm caryophyllene and 1000-4000 ppm nerolidol; 1000-4000 ppm myrcene, 500-5000 ppm linalool, 1000-4500 ppm caryophyllene and 500-4500 ppm nerolidol; or 1000-5500 ppm alpha-pinene, 1000-4000 ppm limonene, 500-4000 ppm linalool and 1000-4500 ppm caryophyllene, and uses in therapy.

### FIELD OF THE INVENTION

The field of art to which this invention generally pertains is terpene compositions, and more specifically to compositions comprising a mixture of terpenes and an oil carrier and uses thereof in therapy.

### BACKGROUND OF THE INVENTION

Terpenes play important roles in cannabinoid-comprising products, affecting the functionality and bioavailability of the cannabinoids and the aroma of the product. Different terpenes are known to have different pharmaceutical effects in combination with cannabinoids. There is a need for compositions comprising different combinations of terpenes and cannabinoids for use in the treatment of specific indications.

### SUMMARY OF THE INVENTION

According to an aspect of some embodiments of the present invention, there is provided a composition comprising a mixture of terpenes consisting of 1000-4500 ppm myrcene, 1000-4000 ppm terpineol, 1000-5500 ppm caryophyllene and 1000-45000 ppm nerolidol; and an oil carrier.

According to a further aspect of some embodiments of the present invention, there is provided a method of treating a sleep disorder (such as insomnia) in a subject in need thereof, the method comprising administering to the subject the composition as disclosed herein.

According to a further aspect of some embodiments of the present invention, there is provided a method of providing neuroprotection to a subject in need thereof, the method comprising administering to the subject the composition as disclosed herein.

According to a further aspect of some embodiments of the present invention, there is provided a composition comprising a mixture of terpenes consisting of 1000-4500 ppm alpha-pinene, 1000-4000 ppm sabinene, 1000-4000 ppm limonene, 500-4000 ppm borneol and 1000-4500 ppm caryophyllene; and an oil carrier.

According to a further aspect of some embodiments of the present invention, there is provided a method of treating a condition selected from the group consisting of pain, neurodegenerative pain, joint pain, muscle pain, joint stiffness, muscle stiffness, depression, anxiety, memory disorder and combinations thereof in a subject in need thereof, the method comprising administering to the subject the composition as disclosed herein.

According to a further aspect of some embodiments of the present invention, there is provided a method of providing neuroprotection in a subject in need thereof, the method comprising administering to the subject the composition as disclosed herein.

According to a further aspect of some embodiments of the present invention, there is provided a composition comprising a mixture of terpenes consisting of 1000-4500 ppm alpha-pinene, 1000-4000 ppm linalool, 1000-4000 ppm terpineol, 1000-4000 ppm borneol, 1000-4500 ppm caryophyllene and 1000-4000 ppm nerolidol; and an oil carrier.

According to a further aspect of some embodiments of the present invention, there is provided a method of treating a symptom selected from the group consisting of agitation, irritability, anxiety, aggression, stress, psychomotor disturbances, memory impairment, cognitive impairment, restlessness, nervousness, self-injury, hyperactivity, impaired social behavior, impaired social communication, a sleep disorder, reduced appetite, anorexia and poor concentration and combinations thereof in a subject in need thereof, the method comprising administering to the subject the composition as disclosed herein.

According to a further aspect of some embodiments of the present invention, there is provided a composition comprising a mixture of terpenes consisting of 1000-4000 ppm myrcene, 500-5000 ppm linalool, 1000-4500 ppm caryophyllene and 500-4500 ppm nerolidol; and an oil carrier.

According to a further aspect of some embodiments of the present invention, there is provided a method of treating a condition selected from the group consisting of a sleep disorder (such as insomnia), agitation, anxiety, restlessness, seizures and combinations thereof in a subject in need thereof, the method comprising administering to the subject the composition as disclosed herein.

According to a further aspect of some embodiments of the present invention, there is provided a composition comprising a mixture of terpenes consisting of 1000-5500 ppm alpha-pinene, 1000-4000 ppm limonene, 500-4000 ppm linalool and 1000-4500 ppm caryophyllene; and an oil carrier.

According to a further aspect of some embodiments of the present invention, there is provided a method of treating a condition selected from the group consisting of anxiety, aggression, agitation, rage attacks, restless, depression, hyperactivity, self-injury, irritability, impaired social behaviors, impaired social communication, seizures and combinations thereof, the method comprising administering to the subject the composition as disclosed herein.

According to a further aspect of some embodiments of the present invention, there is provided a method for improving focus or attention and combinations thereof in a subject in need thereof, the method comprising administering to the subject the composition as disclosed herein.

### DETAILED DESCRIPTION OF THE INVENTION

The present invention, in at least some embodiments, relates to compositions comprising a mixture of terpenes; and an oil carrier, and to uses thereof in therapy.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “cannabinoid” refers to a compound that affects the endocannabinoid system. Cannabinoids are agonists or antagonists to receptors in the endocannabinoid system.

As used herein, the term “THC” refers to THCa (tetrahydrocannabinolic acid) and/or to THC (tetrahydrocannabinol) unless indicated otherwise. As used herein, the term “CBD” refers to CBDa (cannabidiolic acid) and/or to CBD (cannabidiol) unless indicated otherwise. As used herein, the term “CBG” refers to CBGa (cannabigerolic acid) and/or to CBG (cannabigerol) unless indicated otherwise. As used herein, the term “CBN” refers to CBNa (Cannabinolic acid) and/or to CBN (cannabinol) unless indicated otherwise. As used herein, the term “CBC” refers to CBCa (cannabichromenic acid) and/or to CBC (Cannabichromene) unless indicated otherwise. As used herein, the term “CBL” refers to CBLa (Cannabicycol acid) and/or to CBL (Cannabicyclol) unless indicated otherwise. As used herein, the term “THCV” refers to THCVa (tetrahydrocannabivarin acid) and/or to THCV (tetrahydrocannabivarin) unless indicated otherwise. As used herein, the term “CBDV” refers to CBDVa (cannabigerovarin acid).

An oil carrier for use in the present invention may comprise an oil selected from the group consisting of vegetable oils, such as olive oil, hemp oil, soybean oil or sesame oil, or combinations thereof, as well as products thereof, such as, but not limited to, medium-chain triglycerides.

As used herein, the term “treating” includes curing, preventing, ameliorating, mitigating, and reducing the instances of condition, or the symptoms of a condition.

As used herein, the term “administering” includes any mode of administration, such as oral, subcutaneous, sublingual, transmucosal, parenteral, intravenous, intra-arterial, buccal, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the term “impaired social behavior” refers to inappropriate or unexpected behavior in society, such as aggressive response, cursing, undressing, screaming, violence, etc.

As used herein, the term “social behavior” Is intended to encompass all behavior used to interact and communicate with other people. These comprise behavior as simple as looking at another and smiling at them to more complex behavior such as entering into a crowded room with a group of strangers and beginning to interact with them. As used herein, the term “impaired social communication” refers to impaired/abnormal/limited speech or gestures.

As used herein, the term “difficulties in the social use of verbal and nonverbal communication,” includes a lack of ability to alter communication to fit a particular context (e.g., a classroom), to grasp normal rules of conversation, or to understand nonliteral meanings of language.

As used herein, the term “at nighttime” with regard to administration of a composition refers to a composition suitable for administration prior to sleep, preferably prior to a sleep period of at least 5 hours, such as during the hours of darkness. According to some embodiments, a compound suitable for administration at night will have an extended effect of at least 5 hours, such as 5 hours, 6 hours, 7 hours, 8 hours or even more than 8 hours.

As used herein, the term “in daytime” with regard to administration of a composition refers to a composition suitable for administration during the hours in which the subject intends to be awake, such as a time between the subject waking from a night sleep and going to sleep the following night. According to some embodiments, the composition for administration during the day does not cause the subject to become sleepy or dizzy and more preferably increases energy and focus in the subject.

As used herein, the term “elderly” with regard to a subject, refers to a subject of at least 60 years of age.

As used herein, the term “neuroprotection” refers to salvage. recovery, maintenance. protection or regeneration of the nervous system, its cells, structure and function.

As used herein, the term “about” with regard to a numerical value is intended to indication +/−10% of that value, unless otherwise specified.

As used herein, when a numerical value relating to a concentration of a terpene is preceded by the term “about”, the term “about” is intended to indicate +/−30% of that value.

As used herein, when a numerical value relating to a concentration of a cannabinoid is preceded by the term “about”, the term “about” is intended to indicate +/−4% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising a mixture of terpenes consisting of 1000-4500 ppm myrcene, 1000-4000 ppm terpineol, 1000-5500 ppm caryophyllene and 1000-45000 ppm nerolidol; and an oil carrier.

According to some embodiments, the mixture of terpenes further comprises at least one additional terpene selected from the group consisting of 1000-4000 ppm beta-pinene, 1000-4000 ppm sabinene, 1000-4500 ppm borneol, 500-3500 ppm ocimene, 500-4000 ppm linalool, 500-3500 ppm bisabolol and combinations thereof.

According to some embodiments, the at least one additional terpene comprises 1000-4000 ppm beta-pinene, 1000-4000 ppm sabinene, and1000-4500 ppm borneol.

According to some embodiments, the at least one additional terpene comprises 1000-4000 ppm sabinene, 500-3500 ppm ocimene, 500-4000 ppm linalool, and 500-3500 ppm bisabolol. According to some embodiments, the mixture of terpenes consists of 1000-4000 ppm beta-pinene, 1000-4500 ppm myrcene, 1000-4000 ppm terpineol, 1000-4000 ppm borneol, 1500-5500 ppm caryophyllene and 1000-45000 ppm nerolidol.

According to some embodiments, the mixture of terpenes consists of 1000-3000 ppm beta-pinene, 1000-4000 ppm sabinene, 1000-3500 ppm myrcene, 1000-3000 ppm terpineol, 1000-3000 ppm borneol, 1500-4500 ppm caryophyllene and 1000-35000 ppm nerolidol.

According to some embodiments, the mixture of terpenes consists of 500-3500 ppm myrcene, 500-3500 ppm ocimene, 500-4000 ppm linalool, 500-4000 ppm terpineol, 1000-5500 ppm caryophyllene, 1000-45000 ppm nerolidol and 500-3500 ppm bisabolol; and an oil carrier.

According to some embodiments, the mixture of terpenes consists of 1000-4000 ppm sabinene, 500-2500 ppm myrcene, 500-2500 ppm ocimene, 500-3000 ppm linalool, 500-3000 ppm terpineol, 1000-3000 ppm borneol, 1000-4500 ppm caryophyllene, 1000-35000 ppm nerolidol and 500-2500 ppm bisabolol.

According to some embodiments, myrcene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of myrcene to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, terpineol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of terpineol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1. about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1. According to some embodiments, borneol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of borneol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm, about 4500 ppm, about 4600 ppm, about 4700 ppm, about 4800 ppm, about 4900 ppm, about 5000 ppm, about 5100 ppm, about 5200 ppm, about 5300 ppm, about 5400 ppm or about 5500 ppm by weight of the total composition.

According to some embodiments, a ratio of caryophyllene to total cannabinoids (wt/wt) in the composition is in the range of from about 0.015:1 to about 0.01:1, such as about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, nerolidol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of nerolidol to total cannabinoids (wt/wt) in the composition is in the range of is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises at least one selected from the group consisting of 1000-4000 ppm beta-pinene, 1000-4000 ppm sabinene and combinations thereof.

According to some embodiments, beta-pinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of beta-pinene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, sabinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of sabinene to total cannabinoids (wt/wt) in the composition is in the range of from about 0.005:1 to about 0.1:1, such as about 0.005:1, about 0.0075, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to an aspect of some embodiments of the present invention, there is provided a mixture of terpenes consisting of 500-3500 ppm myrcene, 500-3500 ppm ocimene, 500-4000 ppm linalool, 500-4000 ppm terpineol. 1000-5500 ppm caryophyllene, 1000-45000 ppm nerolidol and 500-3500 ppm bisabolol; and an oil carrier.

According to some embodiments, the composition comprises a mixture of terpenes consisting of 500-2500 ppm myrcene, 500-2500 ppm ocimene, 500-3000 ppm linalool, 500-3000 ppm terpineol, 1000-4500 ppm caryophyllene, 1000-35000 ppm nerolidol and 500-2500 ppm bisabolol; and an oil carrier.

According to some embodiments, myrcene is present at about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm or about 3500 ppm by weight of the total composition.

According to some embodiments, a ratio of myrcene to total cannabinoids (wt/wt) in the composition is in the range of from about 0.0025:1 to about 0.1:1, such as about 0.0025:1. about 0.005:1,about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, ocimene is present at about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm or about 3500 ppm by weight of the total composition.

According to some embodiments, a ratio of ocimene to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, linalool is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of linalool to total cannabinoids (wt/wt) in the composition is in the range of from about 0.0025:1 to about 0.1:1, such as about 0.0025:1, about 0.005:1. about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, terpineol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of terpineol to total cannabinoids (wt/wt) in the composition is in the range of from about 0.0025:1 to about 0.1:1, such as about 0.0025:1, about 0.005:1, about 0.0075: 1about 0.01:1, 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm, about 4500 ppm, about 4600 ppm, about 4700 ppm, about 4800 ppm, about 4900 ppm, about 5000 ppm, about 5100 ppm, about 5200 ppm, about 5300 ppm, about 5400 ppm by weight of the total composition.

According to some embodiments, a ratio of caryophyllene to total cannabinoids (wt/wt) in the composition is in the range of from about 0.015:1 to about 0.1:1, such as about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, nerolidol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of nerolidol to total cannabinoids (wt/wt) in the composition is in the range of is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, bisbolol is present at about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm or about 3500 ppm by weight of the total composition.

According to some embodiments, a ratio of bisabolol to total cannabinoids (wt/wt) in the composition is in the range of is in the range of from about 0.005:1 to about 0.1:1, such as about 0.005:1, about 0.0075:1, about 0.01:1, about 0.015:1, about 0.0155:1, about 0.016:1, about 0.0165:1, about 0.017:1, about 0.0175:1, 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.According to some embodiments, the composition further comprises at least one selected form the group consisting of 1000-4000 ppm sabinene, 1000-4500 ppm borneol and combinations thereof.

According to some embodiments, sabinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of sabinene to total cannabinoids (wt/wt) in the composition is in the range of from about 0.0025:1 to about 0.1:1, such as about 0.0025:1, about 0.0055:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, borneol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm, about 4100 ppm, about 4200 ppm, about 4300 ppm, about 4400 ppm or about 4500 by weight of the total composition.

According to some embodiments, a ratio of borneol to total cannabinoids (wt/wt) in the composition is in the range of is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises tetrahydrocannabinol and cannabidiol. According to some such embodiments, the tetrahydrocannabinol and cannabidiol are present at a tetrahydrocannabinol to cannabidiol (T/C) concentration selected from the group consisting of T20/C4, T15/C3, T10/C2, T10/C10, T5/C5, T5/C10, T3/C15, T1/C28 and T1/C20 wt % of the total composition.

According to some embodiments, the composition further comprises cannabidiol at a concentration of from about 2 wt % to about 30 wt % of the total composition, such as about 2 wt %, about 4 wt %, about 6 wt %, about 8 wt %, about 10 wt %, about 12 wt %, about 14 wt %, about 16 wt %, about 18 wt %, about 20 wt %, about 22 wt %, about 24 wt %, about 26 wt %, about 28 wt % or about 30 wt %.

According to some embodiments, the composition is provided in a liquid form. According to some embodiments, the composition is provided in a solid form.

According to some embodiments, the composition is provided in a dosage form suitable for oral administration, such as, for example, as a pill, tablet, capsule, syrup, solution, suspension, powder or the like, or any combination thereof.

According to some embodiments, the composition is provided in a form suitable for administration by inhalation, such as, for example, as a liquid for use in an aerosol, inhaler, nebulizer, vaporizer or the like, or any combination thereof.

According to some alternative embodiments, the composition is provided in a form suitable for parenteral administration, such as for example, for administration by intradermal, subcutaneous, intramuscular, intraosseous, intraperitoneal or intravenous injection, or any combination thereof.

According to some alternative embodiments, the composition is provided in the form of a suppository, such as a vaginal suppository (including a douche, pessary or the like), a rectal suppository, urethral suppository or nasal suppository, or any combination thereof.

According to some embodiments, the composition is for use in treating a sleep disorder.

According to some embodiments, the sleep disorder comprises difficulty in falling asleep, and/or difficulty in staying asleep, intermittent or early waking, such as insomnia.

According to some embodiments, the composition is for use in providing neuroprotection.

According to some embodiments, the composition is for use in reducing anxiety. According to some such embodiments, the composition is for use in treating stress, improving calmness and improving general mood.

According to some embodiments, the composition is for administration to an elderly subject.

According to some embodiments, the composition is for administration at nighttime.

According to an aspect of some embodiments of the present invention, there is provided a method of treating a sleep disorder in a subject in need thereof, the method comprising administering to the subject the composition as disclosed herein. According to some embodiments, the sleep disorder comprises difficulty in falling asleep, and/or difficulty in staying asleep, intermittent or early waking, such as insomnia.

According to an aspect of some embodiments of the present invention, there is provided a method of providing neuroprotection to a subject in need thereof, the method comprising administering to the subject the composition as disclosed herein.

According to some embodiments, there is provided a method of reducing anxiety in a subject in need thereof, the method comprising administering to the subject the composition as disclosed herein. According to some such embodiments, the method is for treating stress, improving calmness and improving general mood.

According to some embodiments of the method, the subject is an elderly subject.

According to some embodiments of the method, administering is performed at nighttime.

According to an aspect of some embodiments of the present invention, there is provided a composition comprising a mixture of terpenes consisting of 1000-4500 ppm alpha-pinene, 1000-4000 ppm sabinene, 1000-4000 ppm limonene, 500-4000 ppm borneol and 1000-4500 ppm caryophyllene; and an oil carrier.

According to some embodiments, alpha-pinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of alpha-pinene to total cannabinoids (wt/wt) in the composition is in the range of is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, sabinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm or about 4000 ppm by weight of the total composition.

According to some embodiments, a ratio of sabinene to total cannabinoids (wt/wt) in the composition is in the range of from about 0.005:1 to 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, limonene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm or about 4000 ppm by weight of the total composition.

According to some embodiments, a ratio of limonene to total cannabinoids (wt/wt) in the composition is in the range of from about 0.005:1 to 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, borneol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm or about 4000 ppm by weight of the total composition.

According to some embodiments, a ratio of borneol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of caryophyllene to total cannabinoids (wt/wt) in the composition is in the range of is in the range of from about 0.005:1 to 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises further comprises at least one selected from the group consisting of 500-2000 ppm terpinolene, 500-3000 ppm eucalyptol and 1000-3000 ppm bisabolol.

According to some embodiments, the composition further comprises at least one selected from the group consisting of 500-3000 ppm terpinolene, 500-4000 ppm eucalyptol and 1000-4000 ppm bisabolol.

According to some embodiments, terpinolene is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm or about 3000 ppm by weight of the total composition.

According to some embodiments, a ratio of terpineol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, eucalyptol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm or about 4000 ppm by weight of the total composition.

According to some embodiments, a ratio of eucalyptol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, bisabolol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 35000 ppm, about 3600 ppm, about 3700 ppm, about 3800 ppm, about 3900 ppm or about 4000 ppm by weight of the total composition.

According to some embodiments, a ratio of bisabolol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the mixture of terpenes consists of 1000-4500 ppm alpha-pinene, 1000-4000 ppm sabinene, 1000-4000 ppm limonene, 500-3000 ppm terpinolene, 500-4000 ppm eucalyptol, 500-4000 ppm borneol and 1000-4500 ppm caryophyllene.

According to some embodiments, the mixture of terpenes consists of 1000-3500 ppm alpha-pinene, 1000-3000 ppm sabinene, 1000-3000 ppm limonene, 500-2000 ppm terpinolene, 500-3000 ppm eucalyptol, 500-3000 ppm borneol and 1000-3500 ppm caryophyllene.

According to some embodiments, the composition further comprises tetrahydrocannabinol and cannabidiol. According to some such embodiments, the tetrahydrocannabinol and cannabidiol are present at a tetrahydrocannabinol to cannabidiol (T/C) concentration selected from the group consisting of T20/C4, T15/C3, T10/C2, T10/C10, T5/C5, T5/C10, T3/C15, T1/C20 and T1/C28 wt % of the total composition.

According to some embodiments, the composition further comprises cannabidiol at a concentration of 30 wt % of the total composition. According to some embodiments, the composition further comprises cannabidiol at a concentration of from about 2 wt % to about 30 wt % of the total composition, such as about 2 wt %, about 4 wt %, about 6 wt %, about 8 wt %, about 10 wt %, about 12 wt %, about 14 wt %, about 16 wt %, about 18 wt %, about 20 wt %, about 22 wt %, about 24 wt %, about 26 wt %, about 28 wt % or about 30 wt %.

According to some embodiments, the composition is provided in a liquid form. According to some embodiments, the composition is provided in a solid form.

According to some embodiments, the composition is provided in a dosage form suitable for oral administration, such as, for example, as a pill, tablet, capsule, syrup, solution, suspension, powder or the like, or any combination thereof.

According to some embodiments, the composition is provided in a form suitable for administration by inhalation, such as, for example, as a liquid for use in an aerosol, inhaler, nebulizer, vaporizer or the like, or any combination thereof.

According to some alternative embodiments, the composition is provided in a form suitable for parenteral administration, such as for example, for administration by intradermal, subcutaneous, intramuscular, intraosseous, intraperitioneal or intravenous injection, or any combination thereof.

According to some alternative embodiments, the composition is provided in the form of a suppository, such as a vaginal suppository (including a douche, pessary or the like), a rectal suppository, urethral suppository or nasal suppository, or any combination thereof.

According to some embodiments, the composition is for use in treating a condition selected from the group consisting of pain, neurodegenerative pain, joint pain, muscle pain, joint stiffness, muscle stiffness, depression, anxiety, memory disorder and combinations thereof.

According to some embodiments, the composition is for use in providing neuroprotection.

According to some embodiments, the composition is for administration to an elderly subject.

According to some embodiments, the composition is for administration in daytime.

According to an aspect of some embodiments of the present invention, there is provided a method of treating a condition selected from the group consisting of pain, neurodegenerative pain, joint pain, muscle pain, joint stiffness, muscle stiffness, depression, anxiety, memory disorder and combinations thereof, the method comprising administering to the subject the composition as disclosed herein.

According to an aspect of some embodiments of the present invention, there is provided a method of providing neuroprotection in a subject in need thereof, the method comprising administering to the subject the composition as disclosed herein.

According to some embodiments of the method, the subject is an elderly subject.

According to some embodiments of the method, administering is performed in daytime.

According to some embodiments, the composition comprises a mixture of terpenes consisting of 1000-4500 ppm alpha-pinene, 1000-4000 ppm linalool, 1000-4000 ppm terpineol, 1000-4000 ppm borneol, 1000-4500 ppm caryophyllene and 1000-4000 ppm nerolidol; and an oil carrier.

According to some embodiments, the composition further comprises at least one selected from the group consisting of 500-3000 ppm beta-pinene and 1000-4000 ppm eucalyptol.

According to some embodiments, the mixture of terpenes consists of 1000-4500 ppm alpha-pinene, 500-3000 ppm beta-pinene, 1000-4000 ppm eucalyptol, 1000-4000 ppm linalool, 1000-4000 ppm terpineol, 1000-4000 ppm borneol, 1000-4500 ppm caryophyllene and 1000-4000 ppm nerolidol.

According to some embodiments, the composition comprises a mixture of terpenes consisting of 1000-3500 ppm alpha-pinene, 500-2000 ppm beta-pinene, 1000-3000 ppm eucalyptol, 1000-3000 ppm linalool, 1000-3000 ppm terpineol, 1000-3000 ppm borneol, 1000-3500 ppm caryophyllene and 1000-3000 ppm nerolidol; and an oil carrier.

According to some embodiments, alpha-pinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of alpha-pinene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, linalool is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of linalool to total cannabinoids (wt/wt) in the composition is in the range of from about 0.005:1 to 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, terpineol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of terpineol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, borneol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of borneol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of caryophyllene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, nerolidol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm. about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of nerolidol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises 500-3000 ppm beta-pinene and/or 1000-4000 ppm eucalyptol.

According to some embodiments, beta-pinene is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 pm, about 2700 ppm, about 2800 ppm, about 2900 ppm or about 3000 ppm by weight of the total composition.

According to some embodiments, a ratio of beta-pinene to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, eucalyptol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of eucalyptol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises tetrahydrocannabinol and cannabidiol. According to some such embodiments, the tetrahydrocannabinol and cannabidiol are present at a tetrahydrocannabinol to cannabidiol (T/C) concentration selected from the group consisting of T20/C4, T15/C3, T10/C2, T10/C10, T5/C5, T5/C10, T3/C15, T1/C20 and T1/C28 wt % of the total composition.

According to some embodiments, the composition further comprises cannabidiol at a concentration of 30 wt % of the total composition. According to some embodiments, the composition further comprises cannabidiol at a concentration of from about 2 wt % to about 30 wt % of the total composition, such as about 2 wt %, about 4 wt %, about 6 wt %, about 8 wt %, about 10 wt %, about 12 wt %, about 14 wt %, about 16 wt %, about 18 wt %, about 20 wt %, about 22 wt %, about 24 wt %, about 26 wt %, about 28 wt % or about 30 wt %.

According to some embodiments, the composition is provided in a liquid form. According to some embodiments, the composition is provided in a solid form.

According to some embodiments, the composition is provided in a dosage form suitable for oral administration, such as, for example, as a pill, tablet, capsule, syrup, solution, suspension, powder or the like, or any combination thereof.

According to some embodiments, the composition is provided in a form suitable for administration by inhalation, such as, for example, as a liquid for use in an aerosol, inhaler, nebulizer, vaporizer or the like, or any combination thereof.

According to some alternative embodiments, the composition is provided in a form suitable for parenteral administration, such as for example, for administration by intradermal, subcutaneous, intramuscular, intraosseous, intraperitioneal or intravenous injection, or any combination thereof.

According to some alternative embodiments, the composition is provided in the form of a suppository, such as a vaginal suppository (including a douche, pessary or the like), a rectal suppository, urethral suppository or nasal suppository, or any combination thereof.

According to some embodiments, the composition is for use in treating a symptom selected from the group consisting of agitation, irritability, anxiety, aggression, stress, psychomotor disturbances, memory impairment, cognitive impairment, restlessness, nervousness, self-injury, hyperactivity, impaired social behavior, impaired social communication, a sleep disorder, reduced appetite, anorexia and poor concentration and combinations thereof.

According to an aspect of some embodiments of the present invention, there is provided a method of treating a symptom selected from the group consisting of agitation, irritability, anxiety, aggression, stress, psychomotor disturbances, memory impairment, cognitive impairment, restlessness, nervousness, self-injury, hyperactivity, impaired social behavior, impaired social communication, a sleep disorder, reduced appetite, anorexia and poor concentration in a subject in need thereof, the method comprising administering to the subject the composition as disclosed herein.

According to some embodiments, the composition is for administration to subject suffering from a condition selected from the group consisting of dementia, an autistic spectrum disorder, an anxiety disorders, psychosis, a psychomotor disturbance, Alzheimer's disease, Parkinson's disease, neuronal damage and combinations thereof.

According to an aspect of some embodiments of the present invention, there is provided a composition comprising a mixture of terpenes consisting of 1000-4000 ppm myrcene, 500-5000 ppm linalool, 1000-4500 ppm caryophyllene and 500-4500 ppm nerolidol; and an oil carrier.

According to some embodiments, myrcene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm or about 4000 ppm by weight of the total composition.

According to some embodiments, a ratio of myrcene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, linalool is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm, about 4500 ppm, about 4600 ppm, about 4700 ppm, about 4800 ppm, about 4900 ppm or about 5000 ppm by weight of the total composition.

According to some embodiments, a ratio of linalool to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of caryophyllene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, nerolidol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400, about 3500, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of nerolidol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises at least one selected from the group consisting of 1000-5000 ppm terpineol, 1000-4000 ppm borneol, 500-3500 ppm alpha-pinene, 500-3000 ppm humulene, and 500-3500 ppm bisabolol.

According to some embodiments, the mixture of terpenes consists of 500-3500 ppm alpha-pinene, 1000-4000 ppm myrcene, 500-5000 ppm linalool, 1000-4500 ppm caryophyllene, 500-35000 ppm nerolidol, 500-3000 ppm humulene, and 500-3500 ppm bisabolol.

According to some embodiments, the mixture of terpenes consists of 500-3500 ppm alpha-pinene, 1000-4000 ppm myrcene, 500-5000 ppm linalool, 1000-4000 ppm terpineol, 1000-4000 ppm borneol, 1000-4500 ppm caryophyllene, 500-3500 ppm nerolidol, 500-3000 ppm humulene, and 500-3500 ppm bisabolol.

According to some embodiments, alpha-pinene is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm or about 3500 ppm by weight of the total composition.

According to some embodiments, a ratio of alpha-pinene to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.According to some embodiments, humulene is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm or about 3000 ppm by weight of the total composition.

According to some embodiments, a ratio of humulene to total cannabinoids (wt/wt) in the composition is in the range of from about 0.0025:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, bisabolol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm or about 3500 ppm by weight of the total composition.

According to some embodiments, a ratio of bisabolol to total cannabinoids (wt/wt) in the composition is in the range of from about 0.0025:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the mixture of terpenes consists of 500-2500 ppm alpha-pinene, 1000-3000 ppm myrcene, 500-4000 ppm linalool, 1000-3500 ppm caryophyllene, 500-25000 ppm nerolidol, 500-2000 ppm humulene, and 500-2500 ppm bisabolol.

According to some embodiments, the mixture of terpenes consists of 500-2500 ppm alpha-pinene, 1000-3000 ppm myrcene, 500-4000 ppm linalool, 1000-5000 ppm terpineol, 1000-4000 ppm borneol, 1000-3500 ppm caryophyllene, 500-25000 ppm nerolidol, 500-2000 ppm humulene, and 500-2500 ppm bisabolol.

According to some embodiments, the mixture of terpenes consists of 500-2500 ppm alpha-pinene, 1000-3000 ppm myrcene, 500-4000 ppm linalool, 1000-3000 ppm terpineol, 1000-3000 ppm borneol, 1000-3500 ppm caryophyllene, 500-25000 ppm nerolidol, 500-2000 ppm humulene, and 500-2500 ppm bisabolol; and an oil carrier.

According to some embodiments, the composition further comprises tetrahydrocannabinol and cannabidiol. According to some such embodiments, the tetrahydrocannabinol and cannabidiol are present at a tetrahydrocannabinol to cannabidiol (T/C) concentration selected from the group consisting T5/C10, T3/C15, T1/C20 and T1/C28 wt % of the total composition.

According to some embodiments, the composition further comprises cannabidiol at a concentration of from about 2 wt % to about 30 wt % of the total composition, such as about 2 wt %, about 4 wt %, about 6 wt %, about 8 wt %, about 10 wt %, about 12 wt %, about 14 wt %, about 16 wt %, about 18 wt %, about 20 wt %, about 22 wt %, about 24 wt %, about 26 wt %, about 28 wt % or about 30 wt %.

According to some embodiments, the composition is provided in a liquid form. According to some embodiments, the composition is provided in a solid form.

According to some embodiments, the composition is provided in a dosage form suitable for oral administration, such as, for example, as a pill, tablet, capsule, syrup, solution, suspension, powder or the like, or any combination thereof.

According to some embodiments, the composition is provided in a form suitable for administration by inhalation, such as, for example, as a liquid for use in an aerosol, inhaler, nebulizer, vaporizer or the like, or any combination thereof.

According to some alternative embodiments, the composition is provided in a form suitable for parenteral administration, such as for example, for administration by intradermal, subcutaneous, intramuscular, intraosseous, intraperitioneal or intravenous injection, or any combination thereof.

According to some alternative embodiments, the composition is provided in the form of a suppository, such as a vaginal suppository (including a douche, pessary or the like), a rectal suppository, urethral suppository or nasal suppository, or any combination thereof.

According to some embodiments, the composition is for use in treating a condition selected from the group consisting of a sleep disorder, agitation, anxiety, restlessness, seizures and combinations thereof. According to some embodiments, the sleep disorder comprises difficulty in falling asleep, and/or difficulty in staying asleep, intermittent or early waking, such as insomnia.

According to some embodiments, the composition is for administration to a subject suffering from an autistic spectrum disorder and/or epilepsy.

According to some embodiments, the composition is for administration at nighttime.

According to an aspect of some embodiments of the present invention, there is provided a method of treating a condition selected from the group consisting of a sleep disorder, agitation, anxiety, restlessness, seizures and combinations thereof in a subject in need thereof, the method comprising administering to the subject the composition as disclosed herein. According to some embodiments of the method, the sleep disorder comprises difficulty in falling asleep, and/or difficulty in staying asleep, intermittent or early waking, such as insomnia.

According to some embodiments of the method, the subject is a subject suffering from an autistic spectrum disorder and/or epilepsy.

According to some embodiments of the method, the administering is performed at nighttime.

According to a further aspect of some embodiments of the present invention, there is provided a composition comprising a mixture of terpenes consisting of 1000-5500 ppm alpha-pinene, 1000-4000 ppm limonene, 500-4000 ppm linalool, and 1000-4500 ppm caryophyllene; and an oil carrier.

According to some embodiments, the composition further comprises at least one selected from the group consisting of 1000-4000 ppm terpineol, 1000-4000 ppm borneol, 500-3000 ppm bisabolol, 1000-4000 ppm beta-pinene and 500-3000 ppm eucalyptol.

According to some embodiments, the composition comprises a mixture of terpenes consisting of 1000-4500 ppm alpha-pinene, 1000-3000 ppm limonene, 500-3000 ppm linalool, 1000-3000 ppm terpineol, 1000-3000 ppm borneol and 1000-3500 ppm caryophyllene; and an oil carrier.

According to some embodiments, the mixture of terpenes consists of 1000-5500 ppm alpha-pinene, 1000-4000 ppm limonene, 500-4000 ppm linalool, 1000-4500 ppm caryophyllene and 500-3000 ppm bisabol.

According to some embodiments, the mixture of terpenes consists of 1000-5500 ppm alpha-pinene, 1000-4000 ppm limonene, 500-4000 ppm linalool, 1000-4000 ppm terpineol, 1000-4000 ppm borneol, 1000-4500 ppm caryophyllene and 500-3000 ppm bisabolol.

According to some embodiments, alpha-pinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm, about 4000 ppm, about 4100 ppm, about 4200 ppm, about 4300 ppm, about 4400 ppm, about 4500 ppm, about 4600 ppm, about 4700 ppm, about 4800 ppm, about 4900 ppm, about 5000 ppm, about 5100 ppm, about 5200 ppm, about 5300 ppm, about 5400 ppm or about 5500 ppm by weight of the total composition.

According to some embodiments, a ratio of alpha-pinene to total cannabinoids (wt/wt) in the composition is in the range of is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, limonene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm or about 4000 ppm by weight of the total composition.

According to some embodiments, a ratio of limonene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, linalool is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm or about 4000 ppm by weight of the total composition.

According to some embodiments, a ratio of linalool to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, caryophyllene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm, about 3500 ppm, about 3600 ppm, about 3700 ppm, about 3800 ppm, about 3900 ppm, about 4000 ppm, about 4100 ppm, about 4200 ppm, about 4300 ppm, about 4400 ppm or about 4500 ppm by weight of the total composition.

According to some embodiments, a ratio of caryophyllene to total cannabinoids (wt/wt) in the composition is in the range of is in the range of from about 0.005:1 to 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises 500-3000 ppm bisabolol.

According to some embodiments, bisabolol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm or about 3000 ppm by weight of the total composition.

According to some embodiments, a ratio of bisabolol to total cannabinoids (wt/wt) in the composition is in the range of from about 0.0025:1 to 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises at least one selected from the group consisting of 1000-4000 ppm beta-pinene and 500-3000 ppm eucalyptol.

According to some embodiments, beta-pinene is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm or about 4000 ppm by weight of the total composition.

According to some embodiments, a ratio of beta-pinene to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, eucalyptol is present at a concentration of about 500 ppm, about 600 ppm, about 700 ppm, about 800 ppm, about 900 ppm, about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm or about 3000 ppm by weight of the total composition.

According to some embodiments, a ratio of eucalyptol to total cannabinoids (wt/wt) in the composition is in the range of from about 0.0025:1 to about 0.1:1, such as about 0.0025:1, about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, terpineol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm or about 4000 ppm by weight of the total composition.

According to some embodiments, a ratio of terpineol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, borneol is present at a concentration of about 1000 ppm, about 1100 ppm, about 1200 ppm, about 1300 ppm, about 1400 ppm, about 1500 ppm, about 1600 ppm, about 1700 ppm, about 1800 ppm, about 1900 ppm, about 2000 ppm, about 2100 ppm, about 2200 ppm, about 2300 ppm, about 2400 ppm, about 2500 ppm, about 2600 ppm, about 2700 ppm, about 2800 ppm, about 2900 ppm, about 3000 ppm, about 3100 ppm, about 3200 ppm, about 3300 ppm, about 3400 ppm about 35000 ppm, about 3600 ppm, about 3700 ppm, about 3800 ppm, about 3900 ppm or about 4000 ppm by weight of the total composition.

According to some embodiments, a ratio of borneol to total cannabinoids (wt/wt) in the composition is in the range of from about 0.005:1 to about 0.1:1, such as about 0.005:1, about 0.0075:1, about 0.01:1, about 0.015:1, about 0.02:1, about 0.025:1, about 0.03:1, about 0.035:1, about 0.04:1, about 0.045:1, about 0.05:1, about 0.055:1, about 0.06:1, about 0.065:1, about 0.07:1, about 0.075:1, about 0.08:1, about 0.085:1, about 0.09:1, about 0.095:1 or about 0.1:1.

According to some embodiments, the composition further comprises tetrahydrocannabinol and cannabidiol. According to some such embodiments, the tetrahydrocannabinol and cannabidiol are present at a tetrahydrocannabinol to cannabidiol (T/C) concentration selected from the group consisting of T5/C10, T3/C15, T1/C20 and T1/C28 wt % of the total composition.

According to some embodiments, the composition further comprises cannabidiol at a concentration of from about 2 wt % to about 30 wt % wt % of the total composition, such as about 2 wt %, about 4 wt %, about 6 wt %, about 8 wt %, about 10 wt %, about 12 wt %, about 14 wt %, about 16 wt %, about 18 wt %, about 20 wt %, about 22 wt %, about 24 wt %, about 26 wt %, about 28 wt % or about 30 wt %.

According to some embodiments, the composition is provided in a liquid form. According to some embodiments, the composition is provided in a solid form.

According to some embodiments, the composition is provided in a dosage form suitable for oral administration, such as, for example, as a pill, tablet, capsule, syrup, solution, suspension, powder or the like, or any combination thereof.

According to some embodiments, the composition is provided in a form suitable for administration by inhalation, such as, for example, as a liquid for use in an aerosol, inhaler, nebulizer, vaporizer or the like, or any combination thereof.

According to some alternative embodiments, the composition is provided in a form suitable for parenteral administration, such as for example, for administration by intradermal, subcutaneous, intramuscular, intraosseous, intraperitioneal or intravenous injection, or any combination thereof.

According to some alternative embodiments, the composition is provided in the form of a suppository, such as a vaginal suppository (including a douche, pessary or the like), a rectal suppository, urethral suppository or nasal suppository, or any combination thereof.

According to some embodiments, the composition is for use in treating a condition selected from the group consisting of anxiety, aggression, agitation, rage attacks, restless, depression, hyperactivity, self-injury, irritability, impaired social behaviors, impaired social communication, seizures and combinations thereof.

According to some embodiments, the composition is for use improving focus and/or attention

According to some embodiments, the composition is for administration to a subject suffering from an autistic spectrum disorder and/or epilepsy.

According to some embodiments, the composition is for administration in daytime.

According to an aspect of some embodiments of the present invention, there is provided a method of treating a condition selected from the group consisting of anxiety, aggression, agitation, rage attacks, restless, depression, hyperactivity, self-injury, irritability, impaired social behaviors, impaired social communication, seizures and combinations thereof, the method comprising administering to the subject the composition as disclosed herein.

According to an aspect of some embodiments of the present invention, there is provided a method for improving focus and/or attention, the method comprising administering to the subject the composition as disclosed herein.

According to some embodiments of the method, the subject is a subject suffering from an autistic spectrum disorder and/or epilepsy.

According to some embodiments of the method, administering is performed in daytime.

### EXAMPLES

The tables below present exemplary compositions comprising specific combinations of terpenes and cannabinoids at various concentrations for use in the treatment of specific indications.

Values shown in the table are terpene concentrations in parts per million (ppm) by weight.

Example 1: Liquid compositions comprising cannabinoid and terpene mixtures for administration at nighttime to an elderly subject for treatment a sleep disorder such as insomnia, or for providing neuroprotection.

Liquid compositions comprising cannabinoids (tetrahydrocannabinol and cannabinol) with terpene mixtures were prepared according to Table 1 below.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition. As used herein, the term “about” with regard to a cannabinoid concentration is intended to refer to a value within ±4% of the given concentration, with the exception of the ratio of T1/C28, wherein “about” refers to a value within ±6% of the given concentration such that the concentrations at this ratio may be in the range of from 0-6% THC and 22-34% CBD.

TABLE 1

| | Cannabinoids content (wt %) | | | |
|---|---|---|---|---|
| | T20/C4, T15/C3, T10/C2, T10/C10, T5/C5, | | | |
| | T5/C10, T3/C15, T1/C28, T1/C20 | | | Terpenes/total |
| Terpenes | Compo- | Compo- | Compo- | cannabinoids |
| (ppm wt) | sition 1 | sition 2 | sition 3 | ratio |
| Myrcene | 1000-4500 | 500-3500 | 1000-4500 | 0.0025-0.0175 |
| Linalool | | 500-4000 | | 0.0025-0.015 |
| Terpineol | 1000-4000 | 500-4000 | 1000-4000 | 0.0025-0.015 |
| Borneol | 1000-4000 | | | 0.005-0.015 |
| Caryo- | 1500-5500 | 1000-5500 | 1500-5500 | 0.015-0.02 |
| phyllene | | | | |
| Nerolidol | 1000-4500 | 1000-4500 | 1000-4500 | 0.005-0.0175 |

Example 2: Solid compositions comprising cannabinoid and terpene mixtures for administration at nighttime to an elderly subject for treatment a sleep disorder such as insomnia, or for providing neuroprotection.

Solid compositions comprising cannabinoid and terpene mixtures according to Table 1 were prepared in tablet form.

Example 3: Compositions comprising cannabidiol and terpene mixtures for administration at nighttime to an elderly subject for treatment a sleep disorder such as insomnia, or for providing neuroprotection.

Solid compositions comprising cannabidiol and terpene mixtures according to Table 2 were prepared in tablet form.

TABLE 2

| | Cannabinoids content (wt %) | | | |
|---|---|---|---|---|
| | T0/C10, T0/C20, T0/C30 | | | Terpenes/total |
| Terpenes | Compo- | Compo- | Compo- | cannabinoids |
| (ppm wt) | sition 1 | sition 2 | sition 3 | ratio |
| Myrcene | 1000-4500 | 500-3500 | 1000-4500 | 0.0025-0.0175 |
| Linalool | | 500-4000 | | 0.0025-0.015 |
| Terpineol | 1000-4000 | 500-4000 | 1000-4000 | 0.0025-0.015 |
| Borneol | 1000-4000 | | | 0.005-0.015 |
| Caryophyllene | 1500-5500 | 1000-5500 | 1500-5500 | 0.015-0.02 |
| Nerolidol | 1000-4500 | 1000-4500 | 1000-4500 | 0.005-0.0175 |

Example 4: Liquid compositions for administration in daytime to an elderly subject for treatment of a condition selected from the group consisting of pain, neurodegenerative pain, joint pain, muscle pain, join and muscle stiffness, depression, anxiety, memory disorders and combinations thereof, or for providing neuroprotection.

Liquid compositions comprising cannabinoids (tetrahydrocannabinol and cannabinol) with terpene mixtures were prepared according to Table 3 below.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition. As used herein, the term “about” with regard to a cannabinoid concentration is intended to refer to a value within ±4% of the given concentration, with the exception of the ratio of T1/C28, wherein “about” refers to a value within ±6% of the given concentration such that the concentrations at this ratio may be in the range of from 0-6% THC and 22-34% CBD.

TABLE 3

Cannabinoids content (wt %)

| | T20/C4, T15/C3, T10/C2, | Terpenes/total |
|---|---|---|
| Terpenes | T10/C10, T5/C5, T5/C10, | cannabinoids |
| (ppm wt) | T3/C15, T1/C20, T1/C28 | ratio |
| Alpha Pinene | 1000-4500 | 0.005-0.0175 |
| Sabinene | 1000-4000 | 0.005-0.015 |
| Limonene | 1000-4000 | 0.005-0.015 |
| Borneol | 500-4000 | 0.0025-0.015 |
| Caryophyllene | 1000-4500 | 0.005-0.0175 |

Example 5: Solid compositions for administration in daytime to an elderly subject for treatment of a condition selected from the group consisting of pain, neurodegenerative pain, joint pain, muscle pain, join and muscle stiffness, depression, anxiety, memory disorders and combinations thereof, or for providing neuroprotection.

Solid compositions comprising cannabinoid and terpene mixtures according to Table 3 were prepared in tablet form.

Example 6: Solid compositions for administration in daytime to an elderly subject for treatment of a condition selected from the group consisting of pain, neurodegenerative pain, joint pain, muscle pain, join and muscle stiffness, depression, anxiety, memory disorders and combinations thereof, or for providing neuroprotection.

Solid compositions comprising cannabidiol and terpene mixtures according to Table 4 were prepared in tablet form.

TABLE 4

Cannabinoids content (wt %)

| | T0/C4, T0/C3, T0/C2, | Terpenes/total |
|---|---|---|
| Terpenes | T0/C5, T0/C10, T0/C15, | cannabinoids |
| (ppm wt) | T0/C20, T0/C28, T0/C24 | ratio |
| Alpha Pinene | 1000-4500 | 0.005-0.0175 |
| Sabinene | 1000-4000 | 0.005-0.015 |
| Limonene | 1000-4000 | 0.005-0.015 |
| Borneol | 500-4000 | 0.0025-0.015 |
| Caryophyllene | 1000-4500 | 0.005-0.0175 |

Example 7: Liquid compositions for administration to a subject suffering from dementia, an autistic spectrum disorder, an anxiety disorder, psychosis, psychomotor disturbance or combinations thereof, for treating a condition selected from the group consisting of agitation, irritability, anxiety, aggression, stress, psychomotor disturbances, memory impairment, cognitive impairment and combinations thereof.

Liquid compositions comprising cannabinoids (tetrahydrocannabinol and cannabinol) with terpene mixtures were prepared according to Table 5 below.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition. As used herein, the term “about” with regard to a cannabinoid concentration is intended to refer to a value within ±4% of the given concentration, with the exception of the ratio of T1/C28, wherein “about” refers to a value within ±6% of the given concentration such that the concentrations at this ratio may be in the range of from 0-6% THC and 22-34% CBD.

TABLE 5

Cannabinoids content (wt %)

| | T20/C4, T15/C3, T10/C2, | Terpenes/total |
|---|---|---|
| Terpenes | T10/C10, T5/C5, T5/C10, | cannabinoids |
| (ppm wt) | T3/C15, T1/C20, T1/C28 | ratio |
| Alpha Pinene | 1000-4500 | 0.005-0.0175 |
| Linalool | 1000-4000 | 0.005-0.015 |
| Terpineol | 1000-4000 | 0.005-0.015 |
| Borneol | 1000-4000 | 0.005-0.015 |
| Caryophyllene | 1000-4500 | 0.005-0.0175 |
| Nerolidol | 1000-4000 | 0.005-0.015 |

Example 2: Solid compositions for administration to a subject suffering from dementia, an autistic spectrum disorder, an anxiety disorder, psychosis, psychomotor disturbance or combinations thereof, for treating a condition selected from the group consisting of agitation, irritability, anxiety, aggression, stress, psychomotor disturbances, memory impairment, cognitive impairment and combinations thereof.

Solid compositions comprising cannabinoid and terpene mixtures according to Table 5 were prepared in tablet form.

Example 8: Solid compositions for administration to a subject suffering from dementia, an autistic spectrum disorder, an anxiety disorder, psychosis, psychomotor disturbance or combinations thereof, for treating a condition selected from the group consisting of agitation, irritability, anxiety, aggression, stress, psychomotor disturbances, memory impairment, cognitive impairment and combinations thereof.

Solid compositions comprising cannabidiol and terpene mixtures according to Table 6 were prepared in tablet form.

TABLE 6

Cannabinoids content (wt %)

| | T0/C4, T0/C3, T0/C2, | Terpenes/total |
|---|---|---|
| Terpenes | T0/C5, T0/C10, T0/C15, | cannabinoids |
| (ppm wt) | T0/C20, T0/C30 | ratio |
| Alpha Pinene | 1000-4500 | 0.005-0.0175 |
| Linalool | 1000-4000 | 0.005-0.015 |
| Terpineol | 1000-4000 | 0.005-0.015 |
| Borneol | 1000-4000 | 0.005-0.015 |
| Caryophyllene | 1000-4500 | 0.005-0.0175 |
| Nerolidol | 1000-4000 | 0.005-0.015 |

Example 9: Liquid compositions for administration at nighttime to a subject suffering from an autistic spectrum disorder or epilepsy, for treatment of a sleep disorder agitation, anxiety, restlessness or seizures.

Liquid compositions comprising cannabinoids (tetrahydrocannabinol and cannabinol) with terpene mixtures were prepared according to Table 7 below.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition. As used herein, the term “about” with regard to a cannabinoid concentration is intended to refer to a value within ±4% of the given concentration, with the exception of the ratio of T1/C28, wherein “about” refers to a value within ±6% of the given concentration such that the concentrations at this ratio may be in the range of from 0-6% THC and 22-34% CBD.

TABLE 7

| | Cannabinoids content (wt %) | | |
|---|---|---|---|
| | | | Terpenes/total |
| Terpenes | T5/C10, T3/C15, | T5/C10, T3/C15, | cannabinoids |
| (ppm wt) | T1/C20, T1/C28 | T1/C20, T1/C28 | ratio |
| Myrcene | 1000-4000 | 1000-4000 | 0.005-0.015 |
| Linalool | 500-5000 | 500-5000 | 0.0025-0.02 |
| Terpineol | | 1000-4000 | 0.005-0.015 |
| Borneol | | 1000-4000 | 0.005-0.015 |
| Caryophyllene | 1000-4500 | 1000-4500 | 0.005-0.0175 |
| Nerolidol | 500-3500 | 500-3500 | 0.0025-0.0125 |

Example 10: Solid compositions for administration at nighttime to a subject suffering from an autistic spectrum disorder or epilepsy, for treatment of a sleep disorder agitation, anxiety, restlessness or seizures.

Solid compositions comprising cannabinoid and terpene mixtures according to Table 7 were prepared in tablet form.

Example 11: Solid compositions for administration at nighttime to a subject suffering from an autistic spectrum disorder or epilepsy, for treatment of a sleep disorder agitation, anxiety, restlessness or seizures.

Solid compositions comprising cannabidiol and terpene mixtures according to Table 8 were prepared in tablet form.

TABLE 8

| | Cannabinoids content (wt %) | | |
|---|---|---|---|
| | | | Terpenes/total |
| Terpenes | T0/C10, T0/C15, | T0/C10, T0/C15, | cannabinoids |
| (ppm wt) | T0/C20, T0/C28 | T0/C20, T0/C28 | ratio |
| Myrcene | 1000-4000 | 1000-4000 | 0.005-0.015 |
| Linalool | 500-5000 | 500-5000 | 0.0025-0.02 |
| Terpineol | | 1000-4000 | 0.005-0.015 |
| Borneol | | 1000-4000 | 0.005-0.015 |
| Caryophyllene | 1000-4500 | 1000-4500 | 0.005-0.0175 |
| Nerolidol | 500-3500 | 500-3500 | 0.0025-0.0125 |

Example 12: Liquid compositions for administration in daytime to a subject suffering from an autistic spectrum disorder and/or epilepsy, for treatment of a condition selected from the group consisting of anxiety, aggression, agitation, rage attacks, restless, depression, hyperactivity, self-injury, irritability, impaired social behaviors, impaired social communication, seizures or combinations thereof, or for improving focus and/or attention

Liquid compositions comprising cannabinoids (tetrahydrocannabinol and cannabinol) with terpene mixtures were prepared according to Table 9 below.

T/C concentrations represent tetrahydrocannabinol (T) to cannabidiol (C) concentration as percentage weight. For example T15/C3 refers to a cannabinoid mixture comprising 15 wt % tetrahydrocannabinol and 3 wt % cannabidiol of the total composition. As used herein, the term “about” with regard to a cannabinoid concentration is intended to refer to a value within ±4% of the given concentration, with the exception of the ratio of T1/C28, wherein “about” refers to a value within ±6% of the given concentration such that the concentrations at this ratio may be in the range of from 0-6% THC and 22-34% CBD.

TABLE 9

| | Cannabinoids content (wt %) | | |
|---|---|---|---|
| Terpenes | T5/C10, T3/C15, | T5/C10, T3/C15, | |
| (ppm wt) | T1/C20, T1/C28 | T1/C20, T1/C28 | |
| Alpha Pinene | 1000-4500 | 1000-4500 | 0.005-0.02 |
| Limonene | 1000-3000 | 1000-3000 | 0.005-0.015 |
| Linalool | 500-3000 | 500-3000 | 0.0025-0.015 |
| Terpineol | | 1000-3000 | 0.005-0.015 |
| Borneol | | 1000-3000 | 0.005-0.015 |
| Caryophyllene | 1000-3500 | 1000-3500 | 0.005-0.0175 |

Example 13: Solid compositions for administration in daytime to a subject suffering from an autistic spectrum disorder and/or epilepsy, for treatment of a condition selected from the group consisting of anxiety, aggression, agitation, rage attacks, restless, depression, hyperactivity, self-injury, irritability, impaired social behaviors, impaired social communication, seizures or combinations thereof, or for improving focus and/or attention

Solid compositions comprising cannabinoid and terpene mixtures according to Table 9 were prepared in tablet form.

Example 14: Solid compositions for administration in daytime to a subject suffering from an autistic spectrum disorder and/or epilepsy, for treatment of a condition selected from the group consisting of anxiety, aggression, agitation, rage attacks, restless, depression, hyperactivity, self-injury, irritability, impaired social behaviors, impaired social communication, seizures or combinations thereof, or for improving focus and/or attention

Solid compositions comprising cannabidiol and terpene mixtures according to Table 10 were prepared in tablet form.

TABLE 10

| | Cannabinoids content (wt %) | | |
|---|---|---|---|
| Terpenes | T0/C10, T0/C15, | T0/C10, T0/C15, | |
| (ppm wt) | T0/C20, T0/C28 | T0/C20, T0/C28 | |
| Alpha Pinene | 1000-5500 | 1000-5500 | 0.005-0.02 |
| Limonene | 1000-4000 | 1000-4000 | 0.005-0.015 |
| Linalool | 500-4000 | 500-4000 | 0.0025-0.015 |
| Terpineol | | 1000-4000 | 0.005-0.015 |
| Borneol | | 1000-4000 | 0.005-0.015 |
| Caryophyllene | 1000-4500 | 1000-4500 | 0.005-0.0175 |

1. A composition comprising a mixture of terpenes consisting of 1000-4500 ppm myrcene, 1000-4000 ppm terpineol, 1000-5500 ppm caryophyllene and 1000-45000 ppm nerolidol; and an oil carrier.

2. The composition of claim 1, wherein said mixture of terpenes further comprises at least one additional terpene selected from the group consisting of 1000-4000 ppm beta-pinene, 1000-4000 ppm sabinene, 1000-4500 ppm borneol 500-3500 ppm ocimene, 500-4000 ppm linalool, 500-3500 ppm bisabolol and combinations thereof.

3-4. (canceled)

5. The composition of claim 1, further comprising tetrahydrocannabinol and/or cannabidiol.

6-11. (canceled)

12. A method of treating a sleep disorder in a subject in need thereof, of providing neuroprotection to a subject in need thereof, or of treating anxiety, the method comprising administering to the subject the composition of claim 1.

13-14. (canceled)

15. The method of claim 12, wherein the subject is an elderly subject.

16. The method of claim 12, wherein said administering is performed at nighttime.

17. A composition comprising a mixture of terpenes consisting of 1000-4500 ppm alpha-pinene, 1000-4000 ppm sabinene, 1000-4000 ppm limonene, 500-4000 ppm borneol and 1000-4500 ppm caryophyllene; and an oil carrier.

18. The composition of claim 17, further comprising at least one selected from the group consisting of 500-3000 ppm terpinolene, 500-4000 ppm eucalyptol and 1000-4000 ppm bisabolol.

19. (canceled)

20. The composition of claim 17, further comprising tetrahydrocannabinol and cannabidiol.

21-25. (canceled)

26. A method of treating a condition selected from the group consisting of pain, neurodegenerative pain, joint pain, muscle pain, joint stiffness, muscle stiffness, depression, anxiety, memory disorder and combinations thereof in a subject in need thereof, or providing neuroprotection in a subject in need thereof, the method comprising administering to the subject the composition of claim 17.

27. (canceled)

28. The method of claim 26, wherein said subject is an elderly subject.

29. The method of claim 26, wherein said administering is performed in daytime.

30. A composition comprising a mixture of terpenes consisting of 1000-4500 ppm alpha-pinene, 1000-4000 ppm linalool, 1000-4000 ppm terpineol, 1000-4000 ppm borneol, 1000-4500 ppm caryophyllene and 1000-4000 ppm nerolidol; and an oil carrier.

31. The composition of claim 30, further comprising at least one selected from the group consisting of 500-3000 ppm beta-pinene and 1000-4000 ppm eucalyptol.

32. The composition of claim 30, wherein said mixture of terpenes consists of 1000-4500 ppm alpha-pinene, 500-3000 ppm beta-pinene, 1000-4000 ppm eucalyptol, 1000-4000 ppm linalool, 1000-4000 ppm terpineol, 1000-4000 ppm borneol, 1000-4500 ppm caryophyllene and 1000-4000 ppm nerolidol.

33. The composition of claim 30, further comprising tetrahydrocannabinol and cannabidiol.

34. The composition of claim 30, further comprising cannabidiol at a concentration of 30 wt % of the total composition.

35-36. (canceled)

37. A method of treating a symptom selected from the group consisting of agitation, irritability, anxiety, aggression, stress, psychomotor disturbances, memory impairment, cognitive impairment, restlessness, nervousness, self-injury, hyperactivity, impaired social behavior, impaired social communication, a sleep disorder, reduced appetite, anorexia and poor concentration in a subject in need thereof, the method comprising administering to the subject the composition of claim 30.

38. The method of claim 37, wherein the subject is suffering from a condition selected from the group consisting of dementia, an autistic spectrum disorder, an anxiety disorder, psychosis, a psychomotor disturbance, Alzheimer's disease, Parkinson's disease, neuronal damage and combinations thereof.

39-64. (canceled)
